(19) United States
(12) Patent Application Publication
(10) Pub. No.: US 2022/0184108 A1
(43) Pub. Date: Jun. 16, 2022

(54) PHARMACEUTICAL AND NUTRITIONAL COMPOSITION TO MAXIMIZE NAD ENZYME'S HEALTH BENEFITS

(71) Applicant: East West Products Corp., Sandy, UT (US)

(72) Inventors: Steve S. Lee, Sandy, UT (US); Francis YM Wong, Singapore (SG); Thee Sheng Tan, Bidor (MY); Siew Seng NG, Singapore (SG)

(73) Assignee: East West Products Corp., Salt Lake City, UT (US)

(21) Appl. No.: 17/119,734

(22) Filed: Dec. 11, 2020

(51) Int. Cl. A61K 31/7076; A61K 31/455; A61K 31/706; A61K 31/7004; A61K 31/015; A61K 31/198; A61K 31/52; A61K 36/02; A61K 31/7032; A61K 36/31; A61K 9/00; A61K 36/87; A61K 36/70; A61K 36/05; A61K 35/748

(52) U.S. Cl. CPC A61K 31/7076; A61K 35/748; A61K 31/706; A61K 31/7004; A61K 31/015; A61K 31/198; A61K 31/52; A61K 36/02; A61K 31/7032; A61K 36/31; A61K 9/0053; A61K 36/87; A61K 36/70; A61K 36/05; A61K 31/455

(57) ABSTRACT

There is a composition for maximizing NAD enzyme's health benefits. The composition includes a nucleoside; the nucleoside is adenosine. The nucleoside is a phosphate derivative; wherein the composition includes 1 to 5 percent weight of adenosine. The composition includes a vitamin B3; wherein the vitamin B3 is in the form of niacin, niacinamide, nicotinic acid or nicotinamide. The composition includes 0.1 to 10 percent weight of the Vitamin B3. The composition includes a nicotinamide mononucleotide. The composition includes a stilbene derivative; wherein the composition includes 0.1 to 5 percent weight stilbene derivative.

### BACKGROUND OF THE INVENTION

### Felid of the Invention

The present invention relates to a composition, specifically a pharmaceutical and nutritional composition to maximize NAD enzyme's health benefits.

### DESCRIPTION OF THE RELATED ART

Nicotinamide adenine dinucleotide (NAD) is a cofactor central to metabolism. Found in all living cells, NAD is called a dinucleotide because it consists of two nucleotides joined through their phosphate groups. One nucleotide may contain an adenine nucleobase and the other nicotinamide. NAD may exist in two forms:

an oxidized and reduced form, abbreviated as NAD⁺ and NADH respectively.

In metabolism, nicotinamide adenine dinucleotide is involved in redox reactions, carrying electrons from one reaction to another. The cofactor is, therefore, found in two forms in cells: NAD⁺ is an oxidizing agent—it accepts electrons from other molecules and becomes reduced. This reaction forms NADH, which may then be used as a reducing agent to donate electrons. These electron transfer reactions are the main function of NAD. However, it is also used in other cellular processes, most notably as a substrate of enzymes in adding or removing chemical groups to or from, respectively, proteins, in posttranslational modifications. Because of the importance of these functions, the enzymes involved in NAD metabolism are targets for drug discovery.

In organisms, NAD may be synthesized from simple building-blocks (de novo) from either tryptophan or aspartic acid, each a case of an amino acid; alternatively, more complex components of the coenzymes are taken up from nutritive compounds such as niacin; similar compounds may be produced by reactions that break down the structure of NAD, providing a salvage pathway that “recycles” them back into their respective active form.

Some NAD may be converted into the coenzyme nicotinamide adenine dinucleotide phosphate (NADP); its chemistry largely parallels that of NAD, though predominantly its role is as a cofactor in anabolic metabolism.

The NAD⁺ chemical species' superscripted addition sign reflects the formal charge on one of its nitrogen atoms; this species' actually a singly charged anion—carrying a (negative) ionic charge of 1—under conditions of physiological pH. NADH, in contrast, is a doubly charged anion, because of its two bridging phosphate groups.

Nicotinamide adenine dinucleotide has several essential roles in metabolism. It acts as a coenzyme in redox reactions, as a donor of ADP-ribose moieties in ADP-ribosylation reactions, as a precursor of the second messenger molecule cyclic ADP-ribose, as well as acting as a substrate for bacterial DNA ligases and a group of enzymes called sirtuins that use NAD⁺ to remove acetyl groups from proteins. In addition to these metabolic functions, NAD⁺ emerges as an adenine nucleotide that may be released from cells spontaneously and by regulated mechanisms, and may therefore have important extracellular roles.

The enzymes that make and use NAD⁺ and NADH are important in both pharmacology and the research into future treatments for disease. Drug design and drug development exploits NAD⁺ in three ways: as a direct target of drugs, by designing enzyme inhibitors or activators based on its structure that change the activity of NAD-dependent enzymes, and by trying to inhibit NAD⁺ biosynthesis.

Because cancer cells utilize increased glycolysis, and because NAD enhances glycolysis, nicotinamide phosphoribosyl transferase (NAD salvage pathway) is often amplified in cancer cells. It has been studied for its potential use in the therapy of neurodegenerative diseases such as Alzheimer's and Parkinson's disease.

NAD⁺ is also a direct target of the drug isoniazid, which is used in the treatment of tuberculosis, an infection caused by Mycobacterium tuberculosis. Isoniazid is a prodrug and once it has entered the bacteria, it is activated by a peroxidase enzyme, which oxidizes the compound into a free radical form. This radical then reacts with NADH, to produce adducts that are very potent inhibitors of the enzymes enoyl-acyl carrier protein reductase, and dihydrofolate reductase.

Since a large number of oxidoreductases use NAD⁺ and NADH as substrates, and bind them using a highly conserved structural motif, the idea that inhibitors based on NAD⁺ could be specific to one enzyme is surprising. However, this may be possible: for example, inhibitors based on the compounds mycophenolic acid and tiazofurin inhibit acid and IMP dehydrogenase at the NAD⁺ binding site. Because of the importance of this enzyme in purine metabolism, these compounds may be useful as anti-cancer, anti-viral, or immunosuppressive drugs. Other drugs are not enzyme inhibitors, but instead activate enzymes involved in NAD⁺ metabolism. Sirtuins are a particularly interesting target for such drugs, since activation of these NAD-dependent deacetylases extends lifespan in some animal models. Compounds such as resveratrol increase the activity of these enzymes, which may be important in their ability to delay aging in both vertebrate, and invertebrate model organisms. In one experiment, mice given NAD for one week had improved nuclear-mitochondrial communication.

Because of the differences in the metabolic pathways of NAD⁺ biosynthesis between organisms, such as between bacteria and humans, this area of metabolism is a promising area for the development of new antibiotics. For example, the enzyme nicotinamidase, which converts nicotinamide to nicotinic acid, is a target for drug design, as this enzyme is absent in humans but present in yeast and bacteria.

In bacteriology, NAD, sometimes referred to factor V, is used a supplement to culture media for some fastidious bacteria.

Some improvements have been made in the field. Examples of references related to the present invention are described below in their own words, and the supporting teachings of each reference are incorporated by reference herein:

U.S. Pat. No. 7,629,329, issued to Lee et al., discloses compositions having an effective amount of Adenosine Triphosphate (“ATP”) sufficient to effect intracellular and extracellular concentrations of ATP in a mammal to improve anaerobic exercise capacity by increasing muscle size and/or strength and methods for using the same. Preferably, a gastric acid secretion inhibitory coating is applied to the effective amount of ATP in a manner that protects the ATP from degradation by gastric juices. ATP compositions of the present invention may be administered in nutraceutical or functional food dosage forms, including oral and non-oral delivery forms. In addition, the effective amount of ATP maybe combined with amino acids, botanicals, functional foods, herbals, nucleotides, nutraceuticals, nutrients, pharmaceuticals, proteins, and/or vitamins in an effort to enhance the targeted activity of the composition.

U.S. Pat. No. 10,392,416, issued to Livingston et al., discloses crystalline forms of a .beta.-nicotinamide mononucleotide, methods of their preparation, and related pharmaceutical preparations thereof. The invention also relates to preparations suitable for nutraceutical, veterinary, and agriculturally-relevant uses.

U.S. Pat. No. 10,307,437, issued to Seals et al., discloses compositions and methods for assessing and treating vascular endothelial dysfunction. Various aspects provide a method for treatment of vascular endothelial dysfunction, comprising administering a composition comprising nicotinamide mononucleotide and a pharmaceutical excipient to a subject. In one embodiment the dose is administered to subjects in response to the indicator. In another embodiment the dose is administered chronically to subjects.

U.S. Pat. No. 10,280,190, issued to Dellinger et al., discloses derivatives of nicotinic acid riboside (NAR), nicotinamide riboside (NR), and reduced NAR and NR, including 1-(2′,3′,5′-triacetyl-beta-D-ribofuranosyl)-1,4-dihydronicotinic acid (4a), and compositions containing the same and/or reduced derivative forms of nicotinamide riboside including 1-(2′,3′,5′-triacetyl-beta-D-ribofuranosyl)-1,4-nicotinamide (2), are provided for use in the care of treatment of skin and skin conditions. In some embodiments, the invention relates to pharmaceutical compositions and cosmetic compositions containing one or more NR, NAR, NRH, or NARH derivatives, or prodrugs or salts thereof. In further embodiments, the invention relates to methods of using one or more NR, NAR, NRH, or NARH derivatives, or prodrugs, solvates, or salts thereof, to promote the increase of intracellular levels of nicotinamide adenine dinucleotide (NAD+) in cells and tissues for improving cell and tissue survival and overall cell and tissue health.

U.S. Patent Application Publication No.: 2006/0229265, by Milburn et al., discloses sirtuin-modulating compounds and methods of use thereof. The sirtuin-modulating compounds may be used for increasing the lifespan of a cell, and treating and/or preventing a wide variety of diseases and disorders including, for example, diseases or disorders related to aging or stress, diabetes, obesity, neurodegenerative diseases, cardiovascular disease, blood clotting disorders, inflammation, cancer, and/or flushing. Also provided are compositions comprising a sirtuin-modulating compound in combination with another therapeutic agent.

The inventions heretofore known suffer from a number of disadvantages which include being ineffective, being limited in use, being expensive, being limited in bio processes, being limited in energy generation, being limited in cell energy, being limited in cellular metabolic pathways, being limited in ways to treat skin conditions, being limited in functionality, being limited in increasing antioxidant levels, being limited in decreasing harmful molecules, being limited in regulating metabolism, being limited in treating thyroid issues, being limited in mitochondria protection, being limited in relieving brain cognition, being limited in treatment of general diseases, being limited in kidney efficiency, being limited in strengthening cardiovascular muscles, and being limited in maintaining brain function.

What is needed is a composition that solves one or more of the problems described herein and/or one or more problems that may come to the attention of one skilled in the art upon becoming familiar with this specification.

### SUMMARY OF THE INVENTION

The present invention has been developed in response to the present state of the art, and in particular, in response to the problems and needs in the art that have not yet been fully solved by currently available compositions. Accordingly, the present invention has been developed to provide a pharmaceutical and nutritional composition to maximize NAD enzyme's health benefits.

According to one embodiment of the invention, there is a composition to maximize NAD enzyme's health benefits that may include methods and compositions related to formulations comprising both active pharmaceutical molecules, prodrugs, and macro/micro nutrients to enhance NAD levels in a human body. NAD enzyme (Nicotinamide adenine dinucleotide) has been demonstrated to improve a large proportion in mitochondrial disease or disorder, insulin resistance, a metabolic syndrome, diabetes, obesity, for increasing insulin sensitivity in diabetic subjects or to treat skin conditions, neuropathy caused by other diseases.

According to one embodiment of the invention, there is a composition for maximizing NAD enzyme's health benefits. The composition may include a nucleoside. The nucleoside may be an adenosine. The nucleoside may be a phosphate derivative; wherein the composition may include 1 to 5 percent weight of adenosine. The composition may include a vitamin B3; wherein the vitamin B3 may be in the form of niacin, niacinamide, nicotinic acid or nicotinamide. The composition may include 0.1 to 10 percent weight of the Vitamin B3. The composition may include a nicotinamide mononucleotide.

The composition may include a stilbene derivative; wherein the composition may include 0.1 to 5 percent weight stilbene derivative. The composition may include a ribose; wherein the composition may include 0.1 to 3 percent weight ribose. The composition may include a vegetable from the cruciferous vegetable family. The composition may include an amino acid. The composition may include an adenine. The composition may include a nucleic acid phosphate. The composition may include an adenosine phosphate. The composition may include an algae. The composition may include a nicotinic acid riboside. The composition may also include a ribose phosphate. The composition may include a nucleic acid.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order for the advantages of the invention to be readily understood, a more particular description of the invention briefly described above will be rendered by reference to specific embodiments that are illustrated in the appended drawing(s). It is noted that the drawings of the invention are not to scale. The drawings are mere schematics representations, not intended to portray specific parameters of the invention. Understanding that these drawing(s) depict only typical embodiments of the invention and are not, therefore, to be considered to be limiting its scope, the invention will be described and explained with additional specificity and detail through the use of the accompanying drawing(s), in which:

FIG. 1 is a perspective view of a NAD molecule composition, according to one embodiment of the invention.

### DETAILED DESCRIPTION OF THE INVENTION

For the purposes of promoting an understanding of the principles of the invention, reference will now be made to the exemplary embodiments illustrated in the drawing(s), and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to an “embodiment,” an “example” or similar language means that a particular feature, structure, characteristic, or combinations thereof described in connection with the embodiment is included in at least one embodiment of the present invention. Thus, appearances of the phrases an “embodiment,” an “example,” and similar language throughout this specification may, but do not necessarily, all refer to the same embodiment, to different embodiments, or to one or more of the figures. Additionally, reference to the wording “embodiment,” “example” or the like, for two or more features, elements, etc. does not mean that the features are necessarily related, dissimilar, the same, etc.

Each statement of an embodiment, or example, is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The features, functions, and the like described herein are considered to be able to be combined in whole or in part one with another as the claims and/or art may direct, either directly or indirectly, implicitly or explicitly.

As used herein, “comprising,” “including,” “containing,” “is,” “are,” “characterized by,” and grammatical equivalents thereof are inclusive or open-ended terms that do not exclude additional unrecited elements or method steps. “Comprising” is to be interpreted as including the more restrictive terms “consisting of” and “consisting essentially of.”

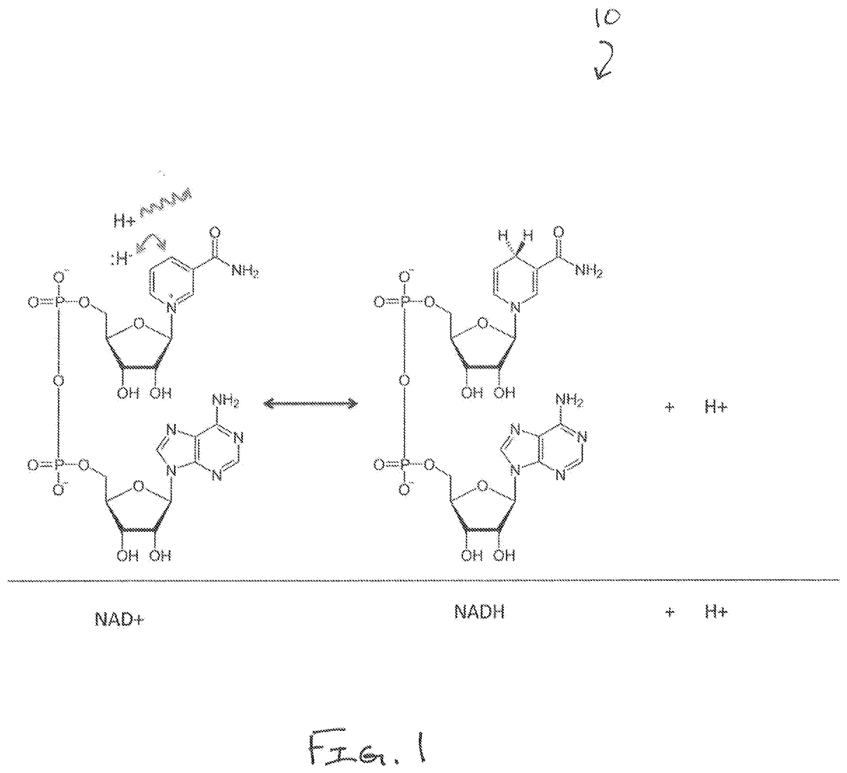

FIG. 1 is a perspective view of a NAD molecule composition, according to one embodiment of the invention. There is shown a NAD molecule composition 10.

According to one embodiment of the invention, there is a composition for maximizing NAD 10 enzyme's health benefits. The composition includes a nucleoside; wherein the nucleoside is adenosine. The nucleoside is a phosphate derivative; wherein the composition includes 1 to 5 percent weight of adenosine. Adenosine is an organic compound that occurs widely in nature in the form of diverse derivatives. The molecule consists of an adenine attached to a ribose via a β-N₉-glycosidic bond. Adenosine is one of four nucleoside building blocks to RNA, which is essential for all life. Its derivatives include the energy carriers adenosine mono-, di-, and triphosphate. Cyclic adenosine monophosphate (cAMP) is pervasive in signal transduction. Adenosyl (Ad) is the radical formed by removal of the 5′-hydroxy (OH) group. Adenosine is found in vitamin B12 and the radical SAM enzymes. Adenosine is also used as a medication.

The composition for maximizing NAD 10 enzyme's health benefits includes a vitamin B3; wherein the vitamin B3 is in the form of niacin, niacinamide, nicotinic acid or nicotinamide. The composition includes 0.1 to 10 percent weight Vitamin B3. Vitamin B₃ is a vitamin family that includes three forms or vitamers:

nicotinamide (niacinamide), niacin (nicotinic acid), and nicotinamide riboside. All three forms of vitamin B₃ are converted within the body to nicotinamide adenine dinucleotide (NAD). NAD is required for human life and people are unable to make it within their bodies without either vitamin B₃ or tryptophan. Nicotinamide riboside was identified as a form of vitamin B₃ in 2004.

The composition for maximizing NAD 10 enzyme's health benefits includes a nicotinamide mononucleotide. Nicotinamide mononucleotide (“NMN”, “NAMN”, and “β-NMN”) is a nucleotide derived from ribose and nicotinamide. NMN is made from B vitamins in the body, and is a molecule naturally occurring in all life forms. Like nicotinamide riboside, NMN is a derivative of niacin, and humans have enzymes that can use NMN to generate nicotinamide adenine dinucleotide (NADH). In mice, NMN enters cells via the small intestines within 10 minutes converting to NAD+ through the Slc12a8 NMN transporter.

Because NADH is a cofactor for processes inside mitochondria, for sirtuins, and for PARP, NMN has been studied in animal models as a potential neuroprotective and anti-aging agent. Dietary supplement companies have aggressively marketed NMN products claiming those benefits. Doses of up to 500 mg was shown safe in men in a recent human study at Keio University School of Medicine, Shinjuku, Tokyo Japan. Multiple long-term human studies are underway.

Nicotinamide riboside (NR) kinase enzymes are essential for exogenously administered utilization of NR and NMN. When administered exogenously, NMN must be converted to NR in order to enter a cell and be re-phosphorylated back to NMN. Both NR and NMN are vulnerable to extracellular degradation by CD38 enzyme.

The composition for maximizing NAD 10 enzyme's health benefits includes a stilbene derivative; wherein the composition includes 0.1 to 5 percent weight stilbene derivative. Stilbene may refer to one of the two stereoisomers of 1,2 diphenylethene: either (E)-Stilbene (trans isomer) or (Z)Stilbene (cis isomer). And a stilbene derivative is a derivative of either (E) or (Z) Stilbene. Stilbene is a mother structure of many phenols and polyphenol in natural vegetables and fruits.

(E)-Stilbene, commonly known as trans-stilbene, is an organic compound represented by the condensed structural formula C₆H₅CH═CHC₆H₅. Classified as a diarylethene, it features a central ethylene moiety with one phenyl group substituents on each end of the carbon-carbon double bond. It has an (E) stereochemistry, meaning that the phenyl groups are located on opposite sides of the double bond, the opposite of its geometric isomer, cis-stilbene. Trans-stilbene occurs as a white crystalline solid at room temperature and is highly soluble in organic solvents. It may be converted to cis-stilbene photochemically, and further reacted to produce phenanthrene.

(Z)-Stilbene is a diarylethene, that is, a hydrocarbon consisting of a cis ethene double bond substituted with a phenyl group on both carbon atoms of the double bond. The name stilbene was derived from the Greek word stilbos, which means shining.

The composition for maximizing NAD 10 enzyme's health benefits includes a ribose; wherein the composition includes 0.1 to 3 percent weight ribose. Ribose is a simple sugar and carbohydrate with molecular formula C₅H₁₀O₅ and the linear-form composition H—(C═O)—(CHOH)₄—H. The naturally-occurring form, d-ribose, is a component of the ribonucleotides from which RNA is built, and so this compound is necessary for coding, decoding, regulation and expression of genes. It has a structural analog, deoxyribose, which is a similarly essential component of DNA. 1-Ribose is an unnatural sugar that was first prepared by Emil Fischer and Oscar Piloty in 1891. It was not until 1909 that Phoebus Levene and Walter Jacobs recognised that d-ribose was a natural product, the enantiomer of Fischer and Piloty's product, and an essential component of nucleic acids. Fischer chose the name “ribose” as it is a partial rearrangement of the name of another sugar, arabinose, of which ribose is an epimer at the 2′ carbon; both names also relate to gum arabic, from which arabinose was first isolated and from which they prepared 1-ribose.

The composition for maximizing NAD 10 enzyme's health benefits includes a vegetable from the cruciferous vegetable family. Cruciferous vegetables are vegetables of the family Brassicaceae (also called Cruciferae) with many genera, species, and cultivars being raised for food production such as cauliflower, cabbage, kale, garden cress, bok choy, broccoli, Brussels sprouts, and similar green leaf vegetables. The family takes its alternative name (Cruciferae, New Latin for “cross-bearing”) from the shape of their flowers, whose four petals resemble a cross.

Ten of the most common cruciferous vegetables eaten by people, known colloquially in North America as cole crops and in the UK and Ireland as brassicas, are in a single species (Brassica oleracea); they are not distinguished from one another taxonomically, only by horticultural category of cultivar groups. Numerous other genera and species in the family are also edible. Cruciferous vegetables are one of the dominant food crops worldwide. They are high in vitamin C and soluble fiber and contain multiple nutrients and phytochemicals.

The composition for maximizing NAD 10 enzyme's health benefits includes an amino acid. Amino acids are organic compounds that contain amine (—NH₂) and carboxyl (—COOH) functional groups, along with a side chain (R group) specific to each amino acid. The key elements of an amino acid are carbon (C), hydrogen (H), oxygen (O), and nitrogen (N), although other elements are found in the side chains of certain amino acids. About 500 naturally occurring amino acids are known (though only 20 appear in the genetic code) and can be classified in many ways. They can be classified according to the core structural functional groups' locations as alpha- (α-), beta- (β-), gamma- (γ-) or delta- (δ-) amino acids; other categories relate to polarity, pH level, and side chain group type (aliphatic, acyclic, aromatic, containing hydroxyl or sulfur, etc.). In the form of proteins, amino acid residues form the second-largest component (water is the largest) of human muscles and other tissues. Beyond their role as residues in proteins, amino acids participate in a number of processes such as neurotransmitter transport and biosynthesis.

The composition for maximizing NAD 10 enzyme's health benefits includes an adenine. Adenine is a nucleobase (a purine derivative). It is one of the four nucleobases in the nucleic acid of DNA that are represented by the letters G-C-A-T. The three others are guanine, cytosine and thymine. Its derivatives have a variety of roles in biochemistry including cellular respiration, in the form of both the energy-rich adenosine triphosphate (ATP) and the cofactors nicotinamide adenine dinucleotide (NAD) and flavin adenine dinucleotide (FAD). It also has functions in protein synthesis and as a chemical component of DNA and RNA. The shape of adenine is complementary to either thymine in DNA or uracil in RNA.

When connected into DNA, a covalent bond is formed between deoxyribose sugar and the bottom left nitrogen (thereby removing the existing hydrogen atom). The remaining structure is called an adenine residue, as part of a larger molecule. Adenosine is adenine reacted with ribose, as used in RNA and ATP; deoxyadenosine is adenine attached to deoxyribose, as used to form DNA.

The composition for maximizing NAD 10 enzyme's health benefits includes a nucleic acid phosphate. Nucleic acid phosphate or nucleic acid metabolism is the process by which nucleic acids (DNA and RNA) are synthesized and degraded. Nucleic acids are polymers of nucleotides. Nucleotide synthesis is an anabolic mechanism generally involving the chemical reaction of phosphate, pentose sugar, and a nitrogenous base. Destruction of nucleic acid is a catabolic reaction. Additionally, parts of the nucleotides or nucleobases can be salvaged to recreate new nucleotides. Both synthesis and degradation reactions require enzymes to facilitate the event. Defects or deficiencies in these enzymes can lead to a variety of diseases.

The composition for maximizing NAD 10 enzyme's health benefits includes an adenosine phosphate. The adenosine phosphate or adenosine monophosphate (AMP), also known as 5′-adenylic acid, is a nucleotide. AMP consists of a phosphate group, the sugar ribose, and the nucleobase adenine; it is an ester of phosphoric acid and the nucleoside adenosine. As a substituent it takes the form of the prefix adenylyl-.

AMP plays an important role in many cellular metabolic processes, being interconverted to ADP and/or ATP. AMP is also a component in the synthesis of RNA. AMP is present in all known forms of life.

The composition for maximizing NAD 10 enzyme's health benefits includes an algae. Algae is an informal term for a large and diverse group of photosynthetic eukaryotic organisms. It is a polyphyletic grouping, including species from multiple distinct clades. Included organisms range from unicellular microalgae, such as Chlorella and the diatoms, to multicellular forms, such as the giant kelp, a large brown alga which may grow up to 50 meters (160 ft) in length. Most are aquatic and autotrophic and lack many of the distinct cell and tissue types, such as stomata, xylem and phloem, which are found in land plants. The largest and most complex marine algae are called seaweeds, while the most complex freshwater forms are the Charophyta, a division of green algae which includes, for example, Spirogyra and stoneworts.

Algae constitute a polyphyletic group since they do not include a common ancestor, and although their plastids seem to have a single origin, from cyanobacteria, they were acquired in different ways. Green algae are examples of algae that have primary chloroplasts derived from endosymbiotic cyanobacteria. Diatoms and brown algae are examples of algae with secondary chloroplasts derived from an endosymbiotic red alga.

Algae exhibit a wide range of reproductive strategies, from simple asexual cell division to complex forms of sexual reproduction. Algae lack the various structures that characterize land plants, such as the phyllids (leaf-like structures) of bryophytes, rhizoids in nonvascular plants, and the roots, leaves, and other organs found in tracheophytes (vascular plants). Most are phototrophic, although some are mixotrophic, deriving energy both from photosynthesis and uptake of organic carbon either by osmotrophy, myzotrophy, or phagotrophy. Some unicellular species of green algae, many golden algae, euglenids, dinoflagellates, and other algae have become heterotrophs (also called colorless or apochlorotic algae), sometimes parasitic, relying entirely on external energy sources and have limited or no photosynthetic apparatus. Some other heterotrophic organisms, such as the apicomplexans, are also derived from cells whose ancestors possessed plastids, but are not traditionally considered as algae. Algae have photosynthetic machinery ultimately derived from cyanobacteria that produce oxygen as a by-product of photosynthesis, unlike other photosynthetic bacteria such as purple and green sulfur bacteria. Fossilized filamentous algae from the Vindhya basin have been dated back to 1.6 to 1.7 billion years ago.

The composition for maximizing NAD 10 enzyme's health benefits includes a nicotinie acid riboside. Nicotinic acid riboside or nicotinamide riboside (NR) is a pyridine-nucleoside similar to vitamin B₃, functioning as a precursor to nicotinamide adenine dinucleotide or NAD+.

The composition for maximizing NAD 10 enzyme's health benefits includes a ribose phosphate. Ribose phosphate or ribose 5-phosphate (R5P) is both a product and an intermediate of the pentose phosphate pathway. The last step of the oxidative reactions in the pentose phosphate pathway is the production of ribulose 5-phosphate. Depending on the body's state, ribulose 5-phosphate can reversibly isomerize to ribose 5-phosphate. Ribulose 5-phosphate can alternatively undergo a series of isomerizations as well as transaldolations and transketolations that result in the production of other pentose phosphates as well as fructose 6-phosphate and glyceraldehyde 3-phosphate (both intermediates in glycolysis). The enzyme ribose-phosphate diphosphokinase converts ribose-5-phosphate into phosphoribosyl pyrophosphate.

The composition for maximizing NAD 10 enzyme's health benefits includes a nucleic acid. Nucleic acids are the biopolymers, or large biomolecules, essential to all known forms of life. The term nucleic acid is the overall name for DNA and RNA. They are composed of nucleotides, which are the monomers made of three components: a 5-carbon sugar, a phosphate group and a nitrogenous base. If the sugar is a compound ribose, the polymer is RNA (ribonucleic acid); if the sugar is derived from ribose as deoxyribose, the polymer is DNA (deoxyribonucleic acid).

Nucleic acids are the most important of all biomolecules. These are found in abundance in all living things, where they function to create and encode and then store information of every living cell of every life-form organism on Earth. In turn, they function to transmit and express that information inside and outside the cell nucleus—to the interior operations of the cell and ultimately to the next generation of each living organism. The encoded information is contained and conveyed via the nucleic acid sequence, which provides the ‘ladder-step’ ordering of nucleotides within the molecules of RNA and DNA.

Strings of nucleotides are bonded to form helical backbones—typically, one for RNA, two for DNA—and assembled into chains of base-pairs selected from the five primary, or canonical, nucleobases, which are: adenine, cytosine, guanine, thymine, and uracil. Thymine occurs only in DNA and uracil only in RNA. Using amino acids and the process known as protein synthesis, the specific sequencing in DNA of these nucleobase-pairs enables storing and transmitting coded instructions as genes. In RNA, base-pair sequencing provides for manufacturing new proteins that determine the frames and parts and most chemical processes of all life forms

In the embodiment the invention relates to methods of using components listed in following to promote the biosynthesis and increase intracellular levels of NAD enzyme in cells and tissues. This can be utilized to improve cell and tissue survival and their physiological functions. Also hundreds of key bio-processes in energy generation, cell survival, cell energy boosted are governed by NAD.

Low level of NAD results in faster aging. In our body, there are two enzymes require NAD enzymatic power to turn on their functions; Poly ADP Ribosepolymerase (PARP), and SIRTs. SIRTs activate several processes controlling aging by regulating blood sugar and fat storage. PARP group is in charge of cell DNA repair. Without that, DNA dysfunction and cell death occurred frequently.

Healthy mitochondria and their functions are smoothly regulated; Mitochondria fusion (protect mitochondria against ros and excessive fragmentation), Mitochondria fission.

Cell renew are normalized. When we are out in the sun, our body is fully invaded by harmful UV lights. Without NAD cell protection activity, we easily get sunburn and more severely skin cancer.

Cellular metabolic pathways to brain, heart, kidneys, and other organs are ameliorated during downward aging process. NAD increases cellular antioxidants level. We are familiar with antioxidants help to scavenge free radicals from our body. Free radicals are harmful molecules that cause diseases and promote aging process.

NAD enhances metabolism and at times thyroid function. Our metabolism is directly governed by NAD and NAD dependent functions. Slightly higher or normal metabolism rate helps to stay away over weight, body fatigue and other thyroid problems.

SIR quality enhanced; gene and DNA protected.

NAD helps maintain brain function. Human brain performs trillions transmission per second. The heavy load of transmission require heavy load of energy. Mitochondria is the power house to facilitate the brain activities. NAD is the key to motivate healthy mitochondria as mentioned above.

Age related diseases and immense human public health problems solved; brain cognition improved, general disease susceptibility lowered, waste cleaning more efficiently in kidneys, cardiovascular muscle strengthened

Biosynthesis of NAD enzyme involves the following pharmaceutical ingredients, prodrugs, micro/macro nutrients, amino acid, and vitamins. These are NR, and its reduced derivatives, Adenine, Ribose, and their Phosphate derivatives, Nicotinamide Mononucleotide, NAR and its reduced derivatives. And others include Amino Acid: Tryptophan. Vitamins: Nicotinic Acid, Niacin, Nicotinamide. Stilbene Derivatives Containing Material: red wine, Polygonaceae herbs, grape skin, skin of tomatoes, dark chocolate. Resveratrol, 3,5,4′-trihydroxy-trans-stilbene and its analogues including 3,4,4′-trihydroxy-trans-Stilbene, 4,4′-dihydroxy-trans-Stilbene. Green vegetables, specifically cruciferous family veggies, which contains Glucoraphanin, Isothiocyanates, Sulforaphane. Foods contain Nicotinamide, Nicotinic Acid: soy (veggie patty, Tofu), yeast, pumpkin seeds, squash seeds, oatmeal, grains, dairy, some algae.

NAD enzyme levels decline during aging has been demonstrated for decades. Dysfunction of NAD homoestasis can be found virtually in all age related diseases including neurodegeneration (mainly dementia, and Parkinson's), diabetes, and cancer. This homeostasis takes charge of body oxidation/reduction enzymes which is crucial for aging process.

NAD enzyme Combats metabolic disturbance and mitochondrial dysfunction mediated through Sirtuin enzymes (SIRT). Together NAD/SIRT has been demonstrated the enhancement of energy metabolism.

Impairment of endothelial NAD signals vascular aging. The deterioration of vascular system causes a decline in capillary density and circulation velocity shown in aging. This is a major cause of mortality and mobility.

Administration of NAD precursors as described in this invention exerts potent anti-aging process. These include vascular effects. Rescuing endothelium-mediated vaso-dilation in the cerebral circulation and improving cerebral blood supply. Another key activity in cerebral circulation is the impacts age-related impairment of endothelial angiogenesis process which in turn plays roles in anti-cancer. In conclusion the composition listed in this invention boost human NAD enzyme involved in aging and chronic diseases in most organs. This in turn relieves and saves the whole public health system.

The following are non-limiting examples and/or prophetic examples of compositions according to one or more embodiments of the invention:

### Example I

In this example, an orally administered elixir is made utilizing purified water as vehicle and following constituents from present invention. 1.0 to 5.0 weight percent of Adenosine, 0.1 to 10.0 weight percent of Vitamin B3 in these forms Niacin, Nicinamide, Nicotinic Acid, or Nicotinamide, 0.1 to 5.0 weight percent of Stilbene derivatives existing in red wine, or herbs belong to Polygonaceae family. Physiological NAD enzyme is synthesized in our body to maintain normal functionality among organs. This blend is intended to people age 30 and above to slow down organs deterioration due to aging process.

### Example 11

1 to 5.0 weight percent of adenine, 0.1 to 5.0 weight percent of Ribose, 0.1 to 3.0 weight percent of red wine concentrates with known concentration of resveratrol. 0.05% to 10.0% weight percent of Nicotinamide or other forms of Vitamin B3. The elixir can be used for subjects 30 year and above to slow down organ aging and maintain normal functionality to maximize the health benefits of NAD enzyme.

### Example III

0.1 to 10.0 weight percent Vitamin B3 in these forms Niacin, Nicinamide, Nicotinic Acid, or Nicotinamide, 0.1 to 10 weight percent of green vegetables specifically cruciferous family veggie. More specifically those contain glucoraphanin, isothiocyanates, and/or sulforaphane. The vegetable includes broccoli, its sprout, cauliflower, kale, brussels sprouts, watercress, mustard leaves. These serve as components for our body to facilitate the bio-synthesis of NAD enzyme. Physiological NAD enzyme is used in our body to maintain normal functionality among organs.

### Example IV

0.1 to 10 weight percent of amino acids specifically Tryptophan, 0.1 to 10.0 weight percent Vitamin B3 in these forms Niacin, Niacinamide, Nicotinic Acid, or Nicotinamide. 0.1 to 5 weight percent Adenine, 0.1 to 5 weight percent ribose are used in the formula to create an elixir. This elixir is used by our body to produce more NAD enzyme. Physiological NAD enzyme is used in our body to maintain normal functionality among human organs.

### Example V

Nucleic Acid Phosphate derivatives 0.1 to 10 weight percent. Stilbene derivatives, including both isomers, 0.1 to 3.0 weight percent. Vitamin B3 derivatives, including Nicotinic Acid, Niacinamide, Nicotinamide, and Niacin, 0.1 to 5.0 weight percent. The combination above serves as raw material for our body to use to synthesize NAD enzyme. Physiological NAD enzyme is used in our body to maintain normal functionality among human organs.

### Example VI

In this example, an orally administered elixir is made utilizing purified water as vehicle and following constituents from present invention. 1.0 to 5.0 weight percent of Adenine, 1.0 to 5.0 weight percent of Ribose derivative as in Ribose Phosphate, 0.1 to 10.0 weight percent of Vitamin B3 in these forms Niacin. Niacinamide, Nicotinic Acid, or Nicotinamide, This formula invention helps our body to produce NAD enzyme, subsequently exerts physiological benefits.

### Example VII

This formula invention combines the bioactive components in the following: 1.0 to 10 weight percents of foods abundant in Vitamin B3 as oatmeal, some algae as green and blue-green algae (also called cyanobacteria), some nuts as in pumpkin seeds, and squash seeds. 0.1 to 5.0 weight percents Adenosine Triphosphate (ATP), 0.1 to 10 weight percents cell nutrients as ribose. This formula invention activates our body mechanism of synthesizing NAD enzyme. This enzyme is demonstrated to control hundreds of key process from energy metabolism to cell aging and functioning.

### Example VIII

0.1 to 5.0 weight percents skin of tomatoes, skin of grapes are used, they are abundant in stilbene derivatives including resveratrol. 0.1 to 10 weight percents algae; chlorella, blue-green algae (Cyanobacteria) are used, They are abundant in Nucleic acids, Vitamin B3 in all forms. In this example, an orally administered elixir is made utilizing purified water as vehicle added with the nutrients mentioned above as formula invention. This formula facilitates production of NAD enzyme in our body. Subsequently, the enzyme ameliorates neural system, improves energy metabolism, and reduces cell ROS accumulation.

### Example IX

0.1 to 10.0 weight percents Nicotinic Acid Riboside or 0.1 to 10 weight percents Niacinamide Riboside or their reduced derivatives. 1.0 to 10.0 weight percent Ribose Phosphate, 0.1 to 10.0 weight percent Nucleic Acid, Adenine for example. In this example, an orally administered elixir is made utilizing purified water as vehicle added with the nutrients mentioned above as formula invention. This combination facilitates the biosynthesis of NAD enzyme in our body.

### Example X

An Elixir Contains the Following Ingredients:

0.1 weight percent to 10.0 weight percent of a combination of NAM, NR, and NA*. 0.1 weight percent to 10.0 weight percent of Nicotinamide Mononucleotide (NMN). 0.1 weight percent to 10.0 weight percent of Ribosyl Phosphate. 0.1 weight percent to 10.0 weight percent of Adenine. * NAM: Nicotinamide. NA: Nicotinic Acid. NR: Nicotinamide Ribose. This elixir serves as precursor of NAD enzyme to be used as essential ingredients to produce the enzyme in our body.

### Example XI

An Elixir Containing the Following Ingredients:

1 to 5 percent weight including a phosphate derivative adenosine. A vitamin B3 component of 0.1 to 10 percent weight selected from the group consisting of niacin, niacinamide, nicotinic acid and nicotinamide. A nicotinamide mononucleotide and a stilbene derivatives component of 0.1 to 5 percent weight. Ribose in an amount of between about 0.1% and 40% by weight. Vegetable from the cruciferous vegetable family in an amount of between about 1% and 50% by weight. An amino acid in an amount of between about 0.1% and 20% by weight. An adenine in an amount of between about 0.1% and 20% by weight. A nucleic acid phosphate in an amount of between about 0.1% and 30% by weight. An adenosine phosphate in an amount of between about 0.1% and 30% by weight. An algae in an amount of between about 0.1% and 50% by weight. A nicotinic acid riboside in an amount of between about 0.1% and 40% by weight. A ribose phosphate in an amount of between about 0.1% and 40% by weight. A nucleic acid in an amount of between about 0.1% and 20% by weight.

### Example XII

An Elixir Containing the Following Ingredients:

1 to 5 percent weight including a phosphate derivative adenosine. A vitamin B3 component of 0.1 to 10 percent weight selected from the group consisting of niacin, niacinamide, nicotinic acid and nicotinamide. A nicotinamide mononucleotide and A stilbene derivatives component of 0.1 to 5 percent weight. Ribose in an amount of between about 5% and 10% by weight. Vegetable from the cruciferous vegetable family in an amount of between about 5% and 10% by weight. An amino acid in an amount of between about 5% and 10% by weight. An adenine in an amount of between about 5% and 10% by weight. A nucleic acid phosphate in an amount of between about 5% and 10% by weight. An adenosine phosphate in an amount of between about 5% and 10% by weight. An algae in an amount of between about 5% and 10% by weight. A nicotinic acid riboside in an amount of between about 5% and 10% by weight. A ribose phosphate in an amount of between about 5% and 10% by weight. A nucleic acid in an amount of between about 5% and 10% by weight.

It is understood that the above-described embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claims rather than by the foregoing description. All changes which come within the meaning and range of equivalency of the claims are to be embraced within their scope.

It is expected that there could be numerous variations of the design of this invention. An example is that the composition may be delivered by an elixir, a syrup, a dried food bar, beverage, mixed beverage, tablet, topical, sublingual, powder, capsule, etc.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims. Further, it is contemplated that an embodiment may be limited to consist of or to consist essentially of one or more of the features, functions, structures, methods described herein.

What is claimed is:

1. A composition for maximizing NAD enzyme's health benefits, comprising:
a) nucleoside including a phosphate derivative adenosine;
b) a form of vitamin B3, selected from the group consisting of niacin, niacinamide, nicotinic acid and nicotinamide
c) nicotinamide mononucleotide; and
c) stilbene derivatives.

2. The composition of claim 1, further comprising ribose in an amount of between about 0.1% and 10% by weight.

3. The composition of claim 1, further comprising a vegetable from the cruciferous vegetable family in an amount of between about 0.1% and 10% by weight.

4. The composition of claim 1, further comprising an amino acid in an amount of between about 0.1% and 10% by weight.

5. The composition of claim 1, further comprising an adenine in an amount of between about 0.1% and 10% by weight.

6. The composition of claim 1, further comprising a nucleic acid phosphate in an amount of between about 0.1% and 10% by weight.

7. The composition of claim 1, further comprising an adenosine phosphate in an amount of between about 0.1% and 10% by weight.

8. The composition of claim 1, further comprising an algae in an amount of between about 0.1% and 10% by weight.

9. The composition of claim 1, further comprising a nicotinic acid riboside in an amount of between about 0.1% and 10% by weight.

10. The composition of claim 1, further comprising a ribose phosphate in an amount of between about 0.1% and 10% by weight.

11. The composition of claim 1, further comprising a nucleic acid in an amount of between about 0.1% and 10% by weight.

12. A composition for maximizing NAD enzyme's health benefits, comprising:
a) a nucleoside component of 1 to 5 percent weight including a phosphate derivative adenosine;
b) a vitamin B3 component of 0.1 to 10 percent weight selected from the group consisting of niacin, niacinamide, nicotinic acid and nicotinamide;
c) nicotinamide mononucleotide; and
d) stilbene derivatives component of 0.1 to 5 percent weight.

13. The composition of claim 12, further comprising a ribose component of 0.1 to 3 percent weight ribose.

14. The composition of claim 13, further comprising a vegetable from the cruciferous vegetable family.

15. The composition of claim 14, further comprising an amino acid.

16. The composition of claim 15, further comprising an adenine.

17. The composition of claim 16, further comprising nucleic acid phosphate.

18. The composition of claim 17, further comprising adenosine phosphate.

19. The composition of claim 18, further comprising:
a) an algae;
b) a nicotinic acid riboside;
c) ribose phosphate; and
d) a nucleic acid.

20. A composition for maximizing NAD enzyme's health benefits, comprising:
a) a phosphate derivative adenosine of 1 to 5 percent weight;
b) a vitamin B3 component of 0.1 to 10 percent weight selected from the group consisting of niacin, niacinamide, nicotinic acid and nicotinamide;
c) a nicotinamide mononucleotide;
d) a stilbene derivative of 0.1 to 5 percent weight;
e) a ribose of 0.1 to 3 percent weight;
f) a vegetable from the cruciferous vegetable family;
g) an amino acid;
h) adenine;
i) a nucleic acid phosphate;
j) adenosine phosphate;
k) an algae;
l) a nicotinic acid riboside;
m) ribose phosphate; and
n) a nucleic acid.
